(19) United States
(12) Patent Application Publication
(10) Pub. No.: US 2026/0284386 A1
(43) Pub. Date: Sep. 24, 2026

(54) ELECTROPORATION/ELECTROCHEMOTHERAPY OF INTERNAL REGIONS WITH LOW INFECTED DEPTH

(71) Applicant: Mohammad ABDOLAHAD, Tehran (IR)

(72) Inventors: Mohammad Abdolahad, Tehran (IR); Mohammad Ali Khayamian, Tehran (IR); Hamed Abadijoo, Tehran (IR); Farshid Rostami Pouria, Tehran (IR); Majid Hasanloo, Tehran (IR); Hossein Simaee, Tehran (IR); Navid Manoochehri, Tehran (IR); Seyed Mojtaba Yazdanparast, Tehran (IR); Alvand Naserghandi, Tehran (IR); Kosar Namakin, Tehran (IR); Seyed Rouhollah Miri, Tehran (IR); Habibollah Mahmoodzadeh, Tehran (IR); Sepideh Mansouri, Tehran (IR)

(73) Assignee: Nano Hesgarsazan Salamat Arya, Tehran, OT (IR)

(21) Appl. No.: 18/689,316

(22) PCT Filed: Dec. 11, 2023
(86) PCT No.: PCT/IB2023/062473

§ 371 Date: Mar. 5, 2024

(51) Int. Cl. A61N 1/32; A61M 25/00; A61N 1/05

(52) U.S. Cl. CPC A61N 1/327; A61M 25/0069; A61M 25/007; A61M 25/0082; A61N 1/05

(57) ABSTRACT

Disclosed herein is a tubular probe for non-invasive electrically stimulation of target cells in an internal target region of a living body. The tubular probe includes a support tube, a first set of electrodes including a plurality of circumferential rings positioned around the support tube in an axially spaced apart relation along the support tube, a second set of electrodes mounted on a head part attached to a distal end of the support tube, and a plurality of electrical connectors connecting the first and second sets of electrodes to an electrical stimulator device. The second set of electrodes includes at least two U-shaped wires separately protruded out from the head part. The first and second sets of electrodes are put inside the internal target region, where an electric field is generated therein due to transferred electrical signals via the first and second sets of electrodes into the internal target region.

### TECHNICAL FIELD

The present disclosure generally relates to methods and apparatus for target cells' ablation and/or delivery of therapeutic agents into a target cell via an electroporation process, and particularly, to an electroporation probe with a structure and an arrangement of electrodes appropriate for applying electroporation to target cells located in sensitive internal regions and/or internal areas of a patient's body which are difficult to access.

### BACKGROUND ART

Minimally invasive treatments are newly emerged methods that have recently been able to replace conventional surgical methods in some cases of cancer treatment, such as methods based on use of ultrasonic waves that destroy tumors by stimulating mechanical waves, or methods based on heat that heat a tumor at a target position and cause it to disappear. Radiofrequency (RF) methods are examples of heating methods in which high frequency electromagnetic waves are used. Each of these methods has prominent features and on the other hand has certain disadvantages, for example, heating-based methods cannot be used near main vessels due to a destruction of intercellular matrix. On the other hand, chemotherapy-based methods also cause problems for a patient. One of the biggest and most common problems in cancer chemotherapy is side effects of drugs on healthy tissues and creation of physiological problems in vital organs of body. Many cancer deaths are due to side effects of chemotherapy drugs on vital organs of body. Today, one of the most important areas of research in cancer treatment is construction of targeted drug delivery systems to tumor areas that cause minimal damage to healthy body tissues.

One of the minimally invasive newly proposed methods is treatment of cancer based on electroporation. In this method, holes are created on cancer cells by applying high voltage electrical pulses, which cause permeability of cancer cells. Holes created by electroporation may have different lifetimes based on their size, and larger holes may last longer. Pore lifetime spans from milliseconds to hours. Holes with long life-times (more than a few hours) may disrupt the tissue homeostasis and result cell death. Depending on an amount of generated electrical field and a duration of electrical pulse, the holes created on cells can be reversible, and as a result, the hole closes after the end of electrical pulse application, and finally, cells will survive. This method is used to deliver more chemotherapy drugs to tumor cells named as electrochemotherapy (ECT) since considerable enhances the efficiency of local chemotherapy. In another case, in which a created hole on cells membrane is such that the cells cannot close it, it is called irreversible electroporation and is done to directly tumor removal without mediation of chemotherapy drugs, which is also called irreversible electroporation (IRE). Depending on type of mass and its location, and many other parameters, treatment can be done in form of ECT or IRE. In this method, high voltage electrical pulses are applied to target cancer cells or cancerous tissue in a short period of time, which leads to a disruption of ionic potential balance of cell membrane; thereby, resulting in generating holes on cells and increasing permeability of cells.

Needle-based electrodes are mostly used for electroporation treatments, and many improvements and efforts have been made to fabricate and utilize electroporation devices having needle electrodes. For example, A. Westersten et al. disclosed in a US patent application numbered as US 2006/0264807 A1 a modular electrode system including a non-symmetrically arranged plurality of needle electrodes, in which a constant-current electrical pulse is applied to the plurality of needle electrodes. The modular electrode system may facilitate delivery of electrical energy to tissues in a manner that assures that the energy dose delivered lies consistently between an upper limit a lower limit; thereby, providing increased electroporation efficiencies. However, conventional electrodes that are mostly utilized in electrochemotherapy have many drawbacks and design weaknesses. For example, conventional needle electrodes cannot be used in sensitive tissues, such as vessels, nerves, intestines, etc. due to bleeding in tissues and/or perforation of vital organs. On the other hand, plate electrodes are not effective in applying electrochemotherapy stimulation due to a lack of a proper interaction surface between tissue and electrode. Moreover, in many cases, a physician is dealing with spaces where it is not possible to apply electrochemotherapy stimulation due to a lack of a proper access.

Hence, there is a need in the art to overcome the problems and drawbacks of electrodes utilizing in an electroporation-based treatment. There is a need in the art for electroporation devices having less-invasive, and preferably, non-invasive electrodes. Specifically, there is a need for a device with electrodes to apply electroporation to cells, which have a structure that minimizes a possibility of damage and bleeding to sensitive tissues (including vascular tissues, nervous tissues, etc.). Also, there is a need for electrodes that allow access to areas that cannot be reached with conventional electrodes. Furthermore, there is a need for electroporation devices having electrodes with an arrangement, size, number, and shape designed and configured to transfer an electric field that can cover wide areas having specific shapes in a living body.

### SUMMARY OF THE DISCLOSURE

This summary is intended to provide an overview of the subject matter of this patent, and is not intended to identify essential elements or key elements of the subject matter, nor is intended to be used to determine the scope of the claimed implementations. The proper scope of this patent may be ascertained from the claims set forth below in view of the detailed description below and the drawings.

In one general aspect, the present disclosure is directed to a tubular probe for non-invasive electrically stimulation of a plurality of target cells in an internal target region of a living body. In an exemplary embodiment, the tubular probe may include a support tube including a distal end and a proximal end, a first set of electrodes including a plurality of circumferential rings positioned around the support tube in an axially spaced apart relation from each other along the support tube, a head part attached to the distal end of the support tube including a second set of electrodes, and a plurality of electrical connectors utilized for connecting the first set of electrodes and the second set of electrodes to an electrical stimulator device. In an exemplary embodiment, the distal end of the support tube may be inserted into the internal target region via guiding the proximal end of the support tube. In an exemplary embodiment, the first set of electrodes may be put in contact with the internal target region. In an exemplary embodiment, the head part may include an electrode substrate engaged to the distal end of the support tube. In an exemplary embodiment, the head part may include at least two apertures. In an exemplary embodiment, the second set of electrodes may include at least two U-shaped wires separately protruded out from the at least two apertures. In an exemplary embodiment, the second set of electrodes may be put in contact with the internal target region.

In an exemplary embodiment, the support tube may include a plurality of openings thereon. In an exemplary embodiment, the plurality of electrical connectors may pass through the support tube. In an exemplary embodiment, the plurality of electrical connectors may include a first set of electrical connectors and a second set of electrical connectors. In an exemplary embodiment, the first set of electrical connectors may be utilized for connecting the first set of electrodes to the electrical stimulator device and the second set of electrical connectors may be utilized for connecting the second set of electrodes to the electrical stimulator device. In an exemplary embodiment, each electrical connector of the plurality of electrical connectors may include an electrically conductive line with a distal end and a proximal end. In an exemplary embodiment, the proximal end of each electrical connector of the plurality of electrical connectors may be in connection with, or capable of connection with, the electrical stimulator device. In an exemplary embodiment, the distal end of each electrical connector of the first set of electrical connectors may be passed through an opening of the plurality of openings and attached to a circumferential ring of the plurality of circumferential rings. In an exemplary embodiment, the distal end of each electrical connector of the first set of electrical connectors may fasten the respective circumferential ring around the support tube. In an exemplary embodiment, the distal end of each electrical connector of the second set of electrical connectors may be attached to a U-shaped wire of the at least two U-shaped wires.

In an exemplary embodiment, the support tube may include an elongated tube made of a biocompatible flexible material. In an exemplary embodiment, the support tube may include a biocompatible polymeric tube. In an exemplary embodiment, the support tube may include a tube with a diameter in a range of 0.5 cm to 3 cm. In an exemplary embodiment, each opening of the plurality of openings may include a hole on the support tube with a diameter in a range of 0.1 cm to 0.5 cm. In an exemplary embodiment, each two adjacent openings may be arranged within a distance in a range of 0.5 cm to 1.5 cm from each other.

In an exemplary embodiment, each electrode of the first set of electrodes may include an electrically conductive biocompatible ring with an internal diameter in a range of 0.5 cm to 3 cm and an external diameter in a range of 0.7 cm to 3.5 cm. In an exemplary embodiment, each U-shaped wire of the at least two U-shaped wires may include a biocompatible electrically conductive wire with a diameter in a range of about 0.5 mm to about 3 mm. In an exemplary embodiment, the at least two U-shaped wires may be arranged in parallel to each other with a distance in a range of 0.5 cm to 1.5 cm between each two adjacent U-shaped wires of the at least two U-shaped wires.

In another general aspect, the present disclosure is directed to a system for in-vivo electroporation of a plurality of target cells in an internal target region of a living body. In an exemplary embodiment, the system may include a tubular probe, an electrical pulse generator electrically connected to the tubular probe, and a processing unit electrically connected to the electrical pulse generator. In an exemplary embodiment, the tubular probe may be utilized to transfer a pulsed electric field to the plurality of target cells.

In an exemplary embodiment, the tubular probe may include a support tube including a distal end and a proximal end, a first set of electrodes including a plurality of circumferential rings positioned around the support tube in an axially spaced apart relation from each other along the support tube, a head part attached to the distal end of the support tube including a second set of electrodes, and a plurality of electrical connectors utilized for connecting the first set of electrodes and the second set of electrodes to the electrical pulse generator. In an exemplary embodiment, the distal end of the support tube may be inserted into the internal target region via guiding the proximal end of the support tube. In an exemplary embodiment, the first set of electrodes may be put in contact with the internal target region. In an exemplary embodiment, the head part may include an electrode substrate engaged to the distal end of the support tube. In an exemplary embodiment, the head part may include at least two apertures. In an exemplary embodiment, the second set of electrodes may include at least two U-shaped wires separately protruded out from the at least two apertures. In an exemplary embodiment, the second set of electrodes may be put in contact with the internal target region.

In an exemplary embodiment, the support tube may include a plurality of openings thereon. In an exemplary embodiment, the plurality of electrical connectors may be passed through the support tube. In an exemplary embodiment, the plurality of electrical connectors may include a first set of electrical connectors and a second set of electrical connectors. In an exemplary embodiment, the first set of electrical connectors may be utilized for connecting the first set of electrodes to the electrical pulse generator and the second set of electrical connectors may be utilized for connecting the second set of electrodes to the electrical pulse generator. In an exemplary embodiment, each electrical connector of the plurality of electrical connectors may include an electrically conductive line with a distal end and a proximal end. In an exemplary embodiment, the proximal end of each electrical connector of the plurality of electrical connectors may be in connection with, or capable of connection with, the electrical pulse generator. In an exemplary embodiment, the distal end of each electrical connector of the first set of electrical connectors may be passed through an opening of the plurality of openings and attached to a circumferential ring of the plurality of circumferential rings. In an exemplary embodiment, the distal end of each electrical connector of the first set of electrical connectors may fasten the respective circumferential ring around the support tube. In an exemplary embodiment, the distal end of each electrical connector of the second set of electrical connectors may be attached to a U-shaped wire of the at least two U-shaped wires.

In an exemplary embodiment, the electrical pulse generator may be utilized to apply a pulsed electric field between at least two electrodes of at least one of the first set of electrodes, the second set of electrodes, and combinations thereof. In an exemplary embodiment, each proximal end of each electrically conductive line of the at least two electrodes may be connected to a different pole of two poles of the electrical pulse generator.

In an exemplary embodiment, the processing unit may include a memory having processor-readable instructions stored therein and a processor. In an exemplary embodiment, the processor may access the memory and execute the processor-readable instructions. In an exemplary embodiment, the processor may be utilized to perform a method when the processor-readable instructions are executed by the processor. In an exemplary embodiment, the method may include inducing electroporation to the plurality of target cells by generating the pulsed electric field between the at least two electrodes of at least two electrodes of at least one of the first set of electrodes, the second set of electrodes, and combinations thereof inside the internal target region via applying at least one sequence of electric voltage pulses between the at least two electrodes utilizing the electrical pulse generator.

In an exemplary embodiment, applying the at least one sequence of electric voltage pulses between the at least two electrodes may include applying at least one sequence of eight square-wave electric voltage pulses with a magnitude in a range of 500 V/cm to 1500 V/cm and a duration of 100 μs between the at least two electrodes.

In an exemplary embodiment, the method may further include generating the pulsed electric field between every two electrodes of at least one of the first set of electrodes, the second set of electrodes, and combinations thereof by periodically changing a connection of at least one of the first set of electrodes, the second set of electrodes, and combinations thereof to the two poles of the electrical pulse generator. In an exemplary embodiment, periodically changing the connection of at least one of the first set of electrodes, the second set of electrodes, and combinations thereof to the two poles of the electrical pulse generator may include periodically substituting connection of at least one electrode of the at least two electrodes by a different electrode of at least one of the first set of electrodes, the second set of electrodes, and combinations thereof. In an exemplary embodiment, substituting connection of the at least one electrode of the at least two electrodes by the different electrode of at least one of the first set of electrodes, the second set of electrodes, and combinations thereof may include electrically disconnecting the at least one electrode of the at least two electrodes from a first pole of the electrical pulse generator and electrically connecting the different electrode of at least one of the first set of electrodes, the second set of electrodes, and combinations thereof to the first pole of the electrical pulse generator. In an exemplary embodiment, periodically changing the connection of at least one of the first set of electrodes, the second set of electrodes, and combinations thereof to the two poles of the electrical pulse generator may include stepwise changing the connection of at least one of the first set of electrodes, the second set of electrodes, and combinations thereof to the two poles of the electrical pulse generator during a plurality of time steps. In an exemplary embodiment, each time step of the plurality of time steps may include a time interval between 0.5 second and 5 seconds.

In an exemplary embodiment, the support tube may include an elongated tube made of a biocompatible flexible material. In an exemplary embodiment, the support tube may include a biocompatible polymeric tube. In an exemplary embodiment, the support tube may include a tube with a diameter in a range of 0.5 cm to 3 cm. In an exemplary embodiment, each opening of the plurality of openings may include a hole on the support tube with a diameter in a range of 0.1 cm to 0.5 cm. In an exemplary embodiment, each two adjacent openings may be arranged within a distance in a range of 0.5 cm to 1.5 cm from each other.

In an exemplary embodiment, each electrode of the first set of electrodes may include an electrically conductive biocompatible ring with an internal diameter in a range of 0.5 cm to 3 cm and an external diameter in a range of 0.7 cm to 3.5 cm. In an exemplary embodiment, each U-shaped wire of the at least two U-shaped wires may include a biocompatible electrically conductive wire with a diameter in a range of about 0.5 mm to about 3 mm. In an exemplary embodiment, the at least two U-shaped wires may be arranged in parallel to each other with a distance in a range of 0.5 cm to 1.5 cm between each two adjacent U-shaped wires of the at least two U-shaped wires.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A schematically shows an exemplary tubular probe, consistent with one or more exemplary embodiments of the present disclosure.

FIG. 1B shows a first exploded view of an exemplary tubular probe, consistent with one or more exemplary embodiments of the present disclosure.

FIG. 1C shows a second exploded view of an exemplary tubular probe, consistent with one or more exemplary embodiments of the present disclosure.

FIG. 1D shows a top view of an exemplary tubular probe, consistent with one or more exemplary embodiments of the present disclosure.

FIG. 1E shows an exemplary system for electrical stimulation of an exemplary target region in a living body using an exemplary tubular probe, consistent with one or more exemplary embodiments of the present disclosure.

FIG. 2 shows an exemplary method for electroporation of an exemplary plurality of target cells, consistent with one or more exemplary embodiments of the present disclosure.

FIG. 3 shows an example computer system in which an embodiment of the present disclosure, or portions thereof, may be implemented as computer-readable code, consistent with one or more exemplary embodiments of the present disclosure.

### DESCRIPTION OF EMBODIMENTS

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

Herein, methods, systems, and devices are disclosed for an electrical treatment (e.g., electroporation) of target cells, particularly cells of a patient's tissues. In an exemplary embodiment, methods and devices may be utilized for target cells' ablation (e.g., tumor cells destruction) and/or delivery of a substance (e.g., a drug, a diagnostic agent, a therapeutic agent, etc.) to exemplary target cells. In an exemplary embodiment, an exemplary device (e.g., a probe) may be disclosed for target cell's electroporation having two or more electrodes designed based on a location, extent, and accessibility of target cells and specific properties of exemplary target cells. In an exemplary embodiment, an exemplary device and electrodes thereof may be designed based on shape, location, and extent of a target area containing exemplary target cells and surrounding areas to be electrically stimulated/treated. In an exemplary embodiment, electrodes of an exemplary device may be non-invasive electrodes while needle electrodes of conventional electroporation devices may be invasive and unsuitable for all parts of a patient's body.

In one general embodiment of the present disclosure, a probe is disclosed. In an exemplary embodiment, an exemplary probe may be utilized for electrical stimulation of a plurality of target cells located in an internal part of a living body. In an exemplary embodiment, an exemplary probe may be utilized for electrical stimulation of a plurality of target cells located in at least one of a tubular area, a cavity-shaped area, or an area with a tubular access path there into. In an exemplary embodiment, an exemplary probe may have a tubular shape adapted to be properly used in tubular and cavity-shaped areas. In an exemplary embodiment, an exemplary probe may include a support tube with two sets of electrodes mounted thereon. In an exemplary embodiment, an exemplary probe may include a first set of electrodes including a plurality of circumferential electrically conductive rings mounted around an outer surface of an exemplary support tube. In an exemplary embodiment, an exemplary probe may further include a second set of electrodes including flat electrically conductive wire blades mounted on a front surface of an exemplary probe. In an exemplary embodiment, a number, size, distance between adjacent electrodes, and other design parameters of each of an exemplary first set of electrodes and an exemplary second set of electrodes may be selected based on a target region containing an exemplary plurality of target cells to be treated.

In an exemplary embodiment, an exemplary probe may be utilized for a non-invasive electrical treatment of a plurality of target cells, which may be located at a hard-to-access location in a living body of a human or an animal. In an exemplary embodiment, an exemplary probe may be utilized for non-invasive electrical treatment of an exemplary plurality of target cells located at areas of an exemplary living body where there is only a tubular access to exemplary areas. In an exemplary embodiment, an exemplary probe may be utilized for a non-invasive electrical treatment of a plurality of target cells located at a location of at least one of at least one of rectum, vagina, colon, esophagus, tracheal, artery, vein, intestine, stomach, bladder, anus entrance, a narrow space inside at least one of digestive, respiratory, urogenital organs, or vessels, and combinations thereof. In an exemplary embodiment, tubular shape and arrangement of an exemplary first set of electrodes and an exemplary second set of electrodes of an exemplary probe may provide generating an electric field all over an exemplary target region; thereby, resulting in a highly effective electrical stimulation of an exemplary target region.

In an exemplary embodiment, an exemplary probe may be utilized for at least one of electrically stimulating of an exemplary plurality of target cells, electroporating an exemplary plurality of target cells, generating an electric field among an exemplary plurality of target cells, electrochemotherapy (ECT) of an exemplary plurality of target cells, electrically ablation of an exemplary plurality of target cells, and combinations thereof. In an exemplary embodiment, an exemplary probe may be utilized for at least one of electrically stimulating of an exemplary plurality of target cells in an area of an exemplary living body, where inserting a needle electrode there into may not be allowed or inserting a needle there may be harmful. As used herein, “target cells” may refer to cells of a part of a living body to be treated by an electrical stimulation (e.g., electroporation); allowing for treatment of exemplary cells including ablating exemplary cells and/or delivery of specific substances (e.g., a drug, a diagnostic agent, a therapeutic agent, etc.) to exemplary cells.

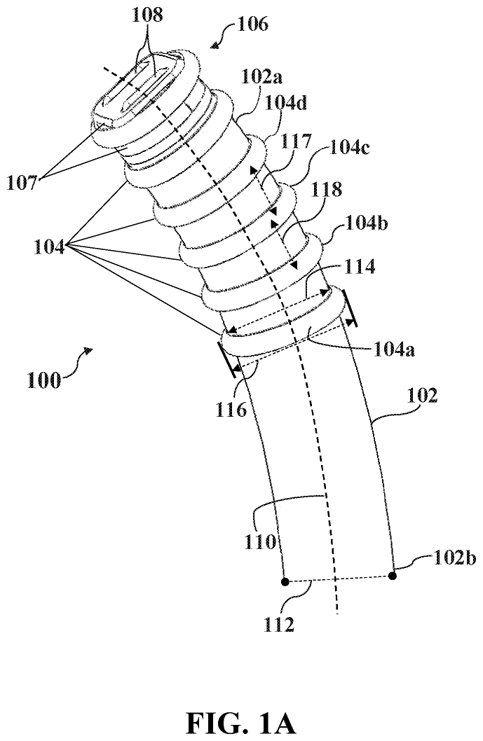

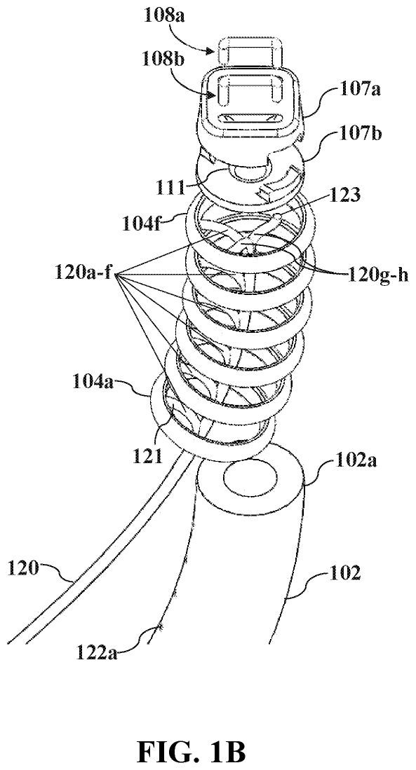

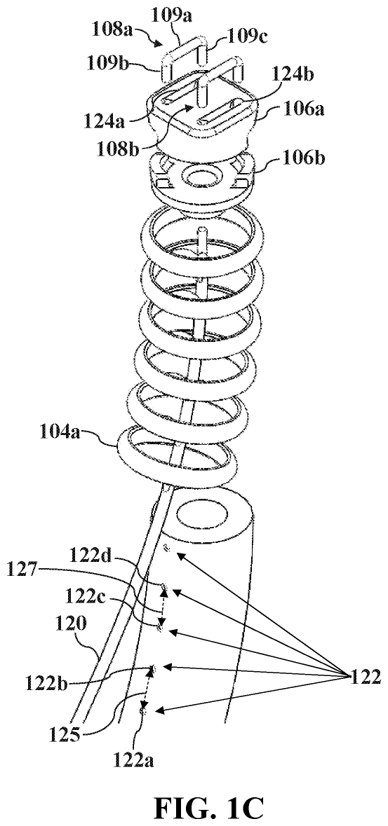

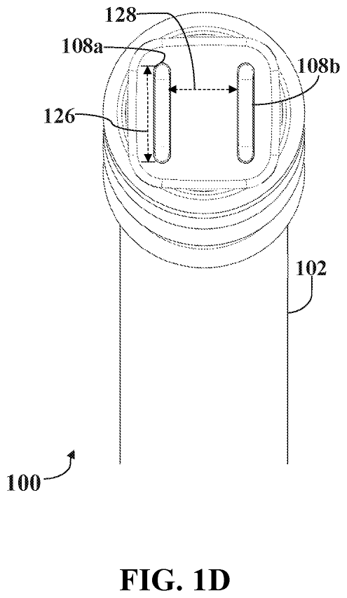

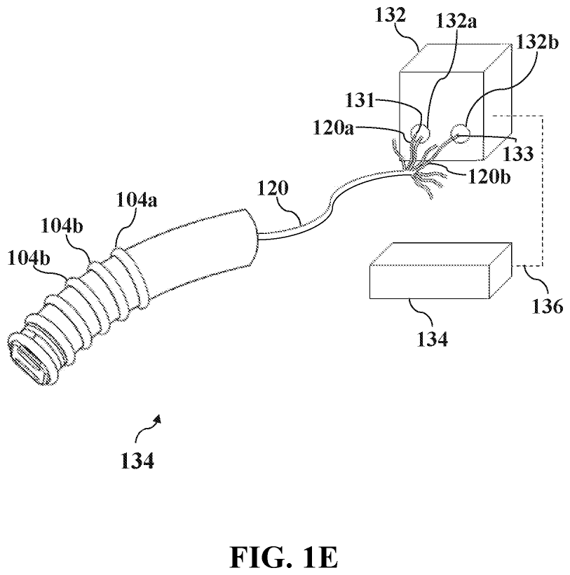

FIG. 1A schematically shows a tubular probe 100, consistent with one or more exemplary embodiments of the present disclosure. In an exemplary embodiment, tubular probe 100 may include a support tube 102, a first set of electrodes 104 positioned around support tube 102, a head part 106 attached to a distal end 102a of support tube 102. In an exemplary embodiment, head part 106 may include a second set of electrodes 108. In an exemplary embodiment, tubular probe 100 may be utilized to be inserted into a living body. In an exemplary embodiment, tubular probe 100 may be utilized to be inserted into a target region of an exemplary living body. In an exemplary embodiment, an exemplary target region may include a plurality of target cells to be treated using tubular probe 100. In an exemplary embodiment, an exemplary target region may include an internal target region inside an exemplary living body. In an exemplary embodiment, tubular probe 100 may be utilized to be inserted into an exemplary target region in the vicinity of a plurality of target cells of an exemplary target region. In an exemplary embodiment, an exemplary plurality of target cells may include a plurality of cancer cells. In an exemplary embodiment, an exemplary plurality of target cells may include at least one of a plurality of cancer cells of a tumor mass, a plurality of remained cancer cells of a resected tumor, a plurality of cancer cells spread throughout an exemplary target region, and combinations thereof. In an exemplary embodiment, an exemplary plurality of target cells may be ablated or destructed using tubular probe 100 through an electrical stimulation treatment. In an exemplary embodiment, tubular probe 100 may be utilized to transfer an electrical signal to an exemplary plurality of target cells to be treated by an exemplary electrical stimulation. In an exemplary embodiment, tubular probe 100 may be utilized to transfer a pulsed electrical signal to an exemplary plurality of target cells to be treated by an electroporation mechanism. In an exemplary embodiment, distal end 102a of support tube 102 may be entered into an exemplary target region, and a location and movement of support tube 102 inside an exemplary target region may be adjusted by guiding a proximal end 102b of support tube 102; allowing for putting first set of electrodes 104 and/or second set of electrodes 108 in contact or in the vicinity of an exemplary plurality of target cells. In an exemplary embodiment, an electric field may be generated among first set of electrodes 104 and/or second set of electrodes 108 in the vicinity of an exemplary plurality of target cells by applying an exemplary electrical signal between two or more electrodes of each of first set of electrodes 104 and/or second set of electrodes 108. In an exemplary embodiment, an exemplary electric field may be generated using an electrical stimulator device (e.g., an electrical pulse generator) electrically connected/coupled to exemplary two or more electrodes first set of electrodes 104 and second set of electrodes 108.

Referring to FIG. 1A, support tube 102 may include an elongated tube made of a biocompatible flexible material. In an exemplary embodiment, support tube 102 may include distal end 102a and proximal end 102b. In an exemplary embodiment, support tube 102 may include a biocompatible polymeric tube. In an exemplary embodiment, support tube 102 may include a tube made of at least one of silicone rubber, polyethylene terephthalate (PET), polydimrthylsiloxane (PDMS), and combinations thereof. In an exemplary embodiment, support tube 102 may include a tube with a diameter 112 in a range of about 0.5 cm to about 3 cm. In an exemplary embodiment, a length of support tube 102 may be varied and changeable based on a length of an exemplary target region where support tube 102 may be inserted therein.

In an exemplary embodiment, first set of electrodes 104 may include a plurality of circumferential rings mounted on support tube 102 surrounding support tube 102. In an exemplary embodiment, each electrode 104a of first set of electrodes 104 may include a ring attached to support tube 102. In an exemplary embodiment, first set of electrodes 104 may be arranged separately circumferentially around support tube 102 along a longitudinal axis 110 of support tube 102. In an exemplary embodiment, first set of electrodes 104 may be positioned around support tube 102 in an axially spaced apart relation from each other along longitudinal axis 110 of support tube 102. In an exemplary embodiment, each electrode 104a may include an electrically conductive biocompatible ring. In an exemplary embodiment, each electrode 104a may include a ring made of stainless steel. In an exemplary embodiment, each electrode 104a may include a ring made of stainless steel 304 or stainless steel 316. In an exemplary embodiment, each electrode 104a may have an internal diameter 114 in a range of about 0.5 cm to about 3 cm and an external diameter 116 in a range of about 0.7 cm to about 3.5 cm. In an exemplary embodiment, each electrode 104a may have internal diameter 114 equal to diameter 112 of support tube 102 and external diameter 116 of about 0.5 cm more than internal diameter 114. In an exemplary embodiment, support tube 102 may include a silicone rubber tube with diameter 112 of about 2 cm and each electrode 104a may include a stainless steel ring with internal diameter 114 of about 2 cm and external diameter 116 of about 2.5 cm mounted around support tube 102. In an exemplary embodiment, each two adjacent electrodes 104b and 104c may be arranged at a distance 118 in a range of about 0.5 cm to about 1.5 cm from each other. In an exemplary embodiment, exemplary distance 118 between exemplary two adjacent electrodes 104b and 104c may be different or equal to a distance 117 between another exemplary two adjacent electrodes 104c and 104d.

FIGS. 1B and 1C show two exploded views of tubular probe 100, consistent with one or more exemplary embodiments of the present disclosure. Furthermore, FIG. 1D shows a top view of tubular probe 100, consistent with one or more exemplary embodiments of the present disclosure. In an exemplary embodiment, head part 106 may be attached to distal end 102a of support tube 102. In an exemplary embodiment, head part 106 may include an electrode substrate 107 engaged to distal end 102a of support tube 102 and second set of electrodes 108 mounted on electrode substrate 107.

In an exemplary embodiment, second set of electrodes 108 may include two or more electrodes. In an exemplary embodiment, second set of electrodes 108 may include two or more U-shaped wires, for example, U-shaped wires 108a and 108b. In an exemplary embodiment, exemplary U-shaped wire 108a may include a flat base 109a with two parallel lateral edges 109b and 109c extending upwards from flat base 109a. In an exemplary embodiment, each U-shaped wire 108a may include a biocompatible electrically conductive wire with a diameter in a range of about 0.5 mm to about 3 mm. In an exemplary embodiment, U-shaped wire 108a may be made of steel. In an exemplary embodiment, U-shaped wire 108a may be made of medical grade stainless steel.

In an exemplary embodiment, electrode substrate 107 may include a top part 107a and a bottom part 107b engaged to distal end 102a of support tube 102. In an exemplary embodiment, top part 107a may include at least two apertures 124a and 124b for mounting U-shaped wires 108a and 108b on electrode substrate 107. In an exemplary embodiment, U-shaped wires 108a and 108b may be protruded out from corresponding apertures 124a and 124b. In an exemplary embodiment, a first lateral edge 109b may pass through a first aperture 124a and a second lateral edge 109c may pass through a second aperture 124b. In an exemplary embodiment, a length in a range of about 0.1 mm to about 0.5 mm of each lateral edge 109b and 109c may be protruded out from top part 107a of electrode substrate 107. Referring to FIG. 1C, flat base 109a of U-shaped wire 108a may have a length 126 in a range of about 0.5 cm to about 1.5 cm. In an exemplary embodiment, second set of electrodes 108 may be arranged parallel to each other. In an exemplary embodiment, each two adjacent U-shaped wires 108a and 108b of second set of electrodes 108 may be in parallel relation to each other with a distance 128 in a range of about 0.5 cm to about 1.5 cm from each other.

In an exemplary embodiment, first set of electrodes 104 may allow for generating an exemplary electric field inside an exemplary living body within a space all around tubular probe 100. Moreover, second set of electrodes 108 may allow for generating an exemplary electric field inside an exemplary living body in front of tubular probe 100. Hence, an exemplary arrangement of first set of electrodes 104 in addition to second set of electrodes 108 may provide covering an exemplary generated electric field within all spaces around tubular probe 100; allowing to ensure all exemplary target cells may be electrically stimulated by adjusting a location of tubular probe 100 inside an exemplary living body during just one insertion of tubular probe 100 into an exemplary living body. In addition, such arrangement of first set of electrodes 104 and second set of electrodes 108 may allow for generating an exemplary electric field non-invasively and exactly at an exemplary target region not stimulating cells of nearby tissues other than an exemplary plurality of target cells.

Referring to FIG. 1B, tubular probe 100 may further include a plurality of electrical connectors 120. In an exemplary embodiment, each electrical connector of plurality of electrical connectors 120 may include an electrically conductive line. In an exemplary embodiment, each electrical connector of plurality of electrical connectors 120 may include an electrically conductive wire. In an exemplary embodiment, each electrical connector of plurality of electrical connectors 120 may connect an electrode of first set of electrodes 104 or second set of electrodes 108 to an exemplary electrical stimulator device. In an exemplary embodiment, plurality of electrical connectors 120 may include a first set of electrical connectors 120a-120f and a second set of electrical connectors 120g and 120h. In an exemplary embodiment, a number of plurality of electrical connectors 120 may be equal to number of electrodes of first set of electrodes 104 in addition to second set of electrodes 108 and not limited to an exemplary number of electrical connectors 120 shown in FIG. 1B. In an exemplary embodiment, plurality of electrical connectors 120 may pass through support tube 102. In an exemplary embodiment, each electrical connector 120a of first set of electrical connectors 120a-120f may have a distal end 121 attached to a corresponding electrode 104a of first set of electrodes 104. In an exemplary embodiment, each electrical connector 120g of second set of electrical connectors 120g and 120h may have a distal end 123 attached to a corresponding electrode 108a of second set of electrodes 108. In an exemplary embodiment, an exemplary electrode 108a may be attached to distal end 123 using at least one of soldering, welding, an irreversible connection, an electrically conductive paste, and combinations thereof.

Referring to FIGS. 1B and 1C, support tube 102 may include a plurality of openings 122 thereon. In an exemplary embodiment, plurality of openings 122 may be used to fasten first set of electrodes 104 around support tube 102. In an exemplary embodiment, first set of electrical connectors 120a-120f may be used to fasten each electrode 104a of first set of electrodes 104 at a location of a corresponding opening 122a of plurality of openings 122. In an exemplary embodiment, each opening 122a of plurality of openings 122 may include a hole on support tube 102 with a diameter in a range of about 0.1 cm to about 0.5 cm. In an exemplary embodiment, each two adjacent openings 122a and 122b of plurality of openings 122 may be arranged within a distance 125 in a range of about 0.5 cm to about 1.5 cm from each other. In an exemplary embodiment, distance 125 between two exemplary adjacent openings 122a and 122b of plurality of openings 122 may be different or equal to a distance 127 between two exemplary adjacent openings 122c and 122d of plurality of openings 122. In an exemplary embodiment, each electrical connector 120a of first set of electrical connectors 120a-120f may be passed through each opening 122a of plurality of openings 122, and distal end 121 of electrical connector 120a may be attached to electrode 104a of first set of electrodes 104. In an exemplary embodiment, an exemplary attachment between distal end 121 and electrode 104a may result in fixing and fastening electrode 104a around support tube 102. In an exemplary embodiment, an exemplary electrode 104a may be attached to distal end 121 using at least one of soldering, welding, an irreversible connection, an electrically paste, and combinations thereof.

In an exemplary embodiment, tubular probe 100 may be utilized for an electrical treatment of an exemplary plurality of target cells located in an exemplary target region of an exemplary living body (i.e., a human, or an animal). In an exemplary embodiment, tubular probe 100 may be utilized for generating an electric field between at least two electrodes of first set of electrodes 104 and/or at least two electrodes of second set of electrodes 108 inside an exemplary target region; allowing for electrically stimulation of an exemplary plurality of target cells. In an exemplary embodiment, tubular probe 100 may be utilized for an exemplary electrical treatment of an exemplary plurality of target cells in an exemplary target region, where an invasive insertion of a needle-like electrode may not be allowed due to a sensitiveness of an exemplary target region. Furthermore, tubular probe 100 may be utilized for an exemplary electrical treatment of an exemplary plurality of target cells in an exemplary target region, where an access there into is limited and an exemplary target region may be accessible only by a flexible tubular structure of tubular probe 101. In an exemplary embodiment, an exemplary target region may include at least one of rectum, vagina, colon, esophagus, tracheal, artery, vein, intestine, stomach, bladder, anus entrance, a narrow space inside at least one of digestive, respiratory, urogenital organs, or vessels, and combinations thereof. In an exemplary embodiment, an exemplary electrical treatment of an exemplary plurality of target cells may include in-vivo electroporation of an exemplary plurality of target cells. In an exemplary embodiment, first set of electrodes 104 and/or second set of electrodes 108 may be utilized for transferring a pulsed electric field from an exemplary electrical pulse generator to an exemplary plurality of target cells while tubular probe 100 is put in contact with an exemplary target region. In an exemplary embodiment, tubular probe 100 may be put in contact with an exemplary target region via insertion of tubular probe 100 into an exemplary living body in the vicinity of an exemplary target region. In an exemplary embodiment, a portion of tubular probe 100 including first set of electrodes 104 and/or second set of electrodes 108 may be inserted into an exemplary target region. In an exemplary embodiment, an electric potential may be applied between at least two electrodes of first set of electrodes 104 and/or second set of electrodes 108 so that an exemplary electric field may be generated in an area including an exemplary plurality of target cells. In an exemplary embodiment, a pulsed electric field may be applied between at least two electrodes of first set of electrodes 104 and/or second set of electrodes 108, and an exemplary pulsed electric field may be generated inside an exemplary target region including an exemplary plurality of target cells. In an exemplary embodiment, an exemplary plurality of target cells of at least one of an exemplary tissue, an exemplary organ, or an exemplary portion thereof may be affected by an exemplary electric field applied between at least two electrodes of first set of electrodes 104 and/or second set of electrodes 108. In an exemplary embodiment, an exemplary plurality of target cells may be electroporated due to an exemplary pulsed electric field applied between at least two electrodes of first set of electrodes 104 and/or second set of electrodes 108. In an exemplary embodiment, an exemplary stimulated plurality of target cells (e.g., a plurality of cancer cells) may be ablated or destructed. In an exemplary embodiment, tubular probe 100 may be utilized for applying a therapeutic method via electrical stimulation (e.g., electroporation) of an exemplary plurality of target cells of an exemplary living body, for example, tumor cells of a cancer patient.

In an exemplary embodiment, an exemplary target region may be swept and scanned by moving tubular probe 100 throughout an exemplary target region and applying an exemplary electric field; thereby, resulting in electrically stimulating (e.g., electroporating) of all exemplary plurality of target cells. In an exemplary embodiment, tubular probe 100 may be moved throughout an exemplary target region by moving proximal end 102b of support tube 102 of tubular probe 100.

In an exemplary embodiment, a system for electroporation including tubular probe 100 may be disclosed. FIG. 1E shows a system 130 for electrical stimulation of an exemplary target region in a living body using tubular probe 100, consistent with one or more exemplary embodiments of the present disclosure. In an exemplary embodiment, system 130 may include tubular probe 100, an electrical stimulator device 132, and a processing unit 134. In an exemplary embodiment, electrical stimulator device 132 may include a power supply or an electrical voltage generator. In an exemplary embodiment, electrical stimulator device 132 may include an electrical pulse generator. In an exemplary embodiment, electrical stimulator device 132 may include an electroporation pulse generator.

In an exemplary embodiment, tubular probe 100 may be electrically connected to electrical stimulator device 132 via plurality of electrical connectors 120. In an exemplary embodiment, two or more electrodes of first set of electrodes 104 and/or second set of electrodes 108 of tubular probe 100 may be electrically connected to electrical stimulator device 132 via two or more respective electrical connectors of plurality of electrical connectors 120. In an exemplary embodiment, at least one electrical connector 120a of plurality of electrical connectors 120 attached to a corresponding electrode 104a may have a proximal end 131 connected to a first pole 132a of electrical stimulator device 132, and at least another electrical connector 120b of plurality of electrical connectors 120 attached to a corresponding electrode 104b may have a proximal end 133 connected to a second pole 132b of electrical stimulator device 132. In an exemplary embodiment, electrical stimulator device 132 may be utilized to apply an electrical signal between one or more pairs of first set of electrodes 104 and/or second set of electrodes 108 while first set of electrodes 104 and/or second set of electrodes 108 being put in contact with an exemplary target region in an exemplary living body including an exemplary plurality of target cells; therefore, an electric field within an exemplary target region may be generated and an exemplary plurality of target cells may be electrically stimulated. For example, electrical stimulator device 132 may be utilized to apply an exemplary electrical signal between electrode 104a and electrode 104b; thereby, resulting in generating an electric field in an area of an exemplary target region between and around two electrodes 104a and 104b.

Referring to FIG. 1E, In an exemplary embodiment, processing unit 134 may be electrically connected to electrical stimulator device 132 via a wireless connection or utilizing respective electrically conductive wire 136. In an exemplary embodiment, processing unit 124 may include a memory having processor-readable instructions stored therein and a processor. In an exemplary embodiment, an exemplary processor may be utilized to access an exemplary memory and execute exemplary processor-readable instructions. In an exemplary embodiment, executing exemplary processor-readable instructions by an exemplary processor may configure an exemplary processor to perform a method. In an exemplary embodiment, an exemplary method may include applying a therapeutical treatment to an exemplary plurality of target cells inside an exemplary living body by electrically stimulating an exemplary plurality of target cells. In an exemplary embodiment, an exemplary method may include at least one of treating an exemplary plurality of target cells, ablating an exemplary plurality of target cells, delivering a drug or therapeutical substance to an exemplary plurality of target cells, and combinations thereof by electroporation an exemplary plurality of target cells.

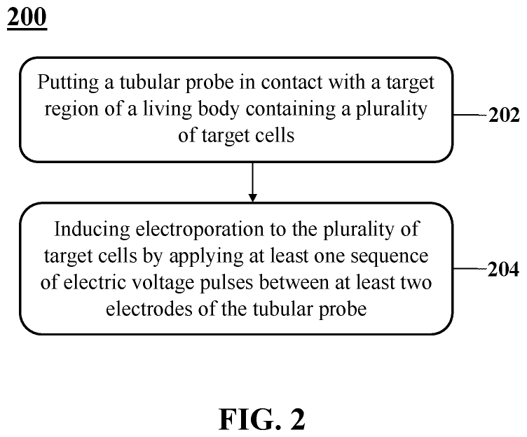

FIG. 2 shows a method 200 for electroporation of an exemplary plurality of target cells, consistent with one or more exemplary embodiments of the present disclosure. In an exemplary embodiment, method 200 may include putting tubular probe 100 in contact with an exemplary target region of an exemplary living body containing an exemplary plurality of target cells (step 202) and inducing electroporation to an exemplary plurality of target cells by generating an exemplary pulsed electric field between at least two electrodes of at least one of first set of electrodes 104, second set of electrodes 108, and combinations thereof of tubular probe 100 inside an exemplary target region (step 204). In an exemplary embodiment, method 200 may be carried out utilizing tubular probe 100 and system 130. So, method 200 may be described herein below in connection with FIGS. 1A-1E.

In further detail with respect to step 202, tubular probe 100 may be put in contact with an exemplary target region of an exemplary living body, where an exemplary target region may contain an exemplary plurality of target cells. In an exemplary embodiment, an exemplary target region may include an internal region inside an exemplary living body. In an exemplary embodiment, an exemplary target region and/or an access path thereto may include a tubular-shaped or cavity-shaped area inside an exemplary living body. In an exemplary embodiment, an exemplary target region may include at least one of rectum, vagina, colon, esophagus, tracheal, artery, vein, intestine, stomach, bladder, anus entrance, a narrow space inside at least one of digestive, respiratory, urogenital organs, or vessels, and combinations thereof.

In an exemplary embodiment, putting tubular probe 100 in contact with an exemplary target region of an exemplary living body may include inserting tubular probe 100 into an exemplary living body in the vicinity of an exemplary target region. In an exemplary embodiment, putting tubular probe 100 in contact with an exemplary target region of an exemplary living body may include inserting tubular probe 100 into an exemplary living body inside an exemplary target region. In an exemplary embodiment, putting tubular probe 100 in contact with an exemplary target region of an exemplary living body may include placing a portion of tubular probe 100 including at least one of first set of electrodes 104, second set of electrodes 108, and combinations thereof in the vicinity of or inside an exemplary target region.

In an exemplary embodiment, putting tubular probe 100 in contact with an exemplary target region may include placing tubular probe 100 in the vicinity or in contact with an exemplary plurality of target cells to be treated. In an exemplary embodiment, putting tubular probe 100 in contact with an exemplary target region may include sweeping or scanning an exemplary target region by tubular probe 100 via moving proximal end 102b while conducting step 204 of method 200.

In further detail with respect to step 204, step 204 may include inducing electroporation to an exemplary plurality of target cells by generating an exemplary pulsed electric field between at least two electrodes of at least one of first set of electrodes 104, second set of electrodes 108, and combinations thereof of tubular probe 100 inside an exemplary target region. In an exemplary embodiment, step 204 may include inducing electroporation to an exemplary plurality of target cells by generating an exemplary pulsed electric field between at least two electrodes of at least one of first set of electrodes 104, second set of electrodes 108, and combinations thereof of tubular probe 100 inside an exemplary target region via applying at least one sequence of electric voltage pulses between exemplary at least two electrodes utilizing electrical pulse generator 132.

In an exemplary embodiment, step 204 may include applying at least one sequence of electric voltage pulses between at least two exemplary electrodes 104a and 104b of first set of electrodes 104 and/or at least two exemplary electrodes 108a and 108b of second set of electrodes 108. In an exemplary embodiment, applying at least one sequence of electric voltage pulses between at least two exemplary electrodes 104a and 104b (and/or two exemplary electrodes 108a and 108b) may include applying at least one sequence of eight square-wave electric voltage pulses with a magnitude in a range of about 500 V/cm to about 1500 V/cm and a duration of about 100 μs between at least two exemplary electrodes 104a and 104b (and/or two exemplary electrodes 108a and 108b). In an exemplary embodiment, applying at least one sequence of electric voltage pulses between at least two exemplary electrodes 104a and 104b (and/or two exemplary electrodes 108a and 108b) may include applying at least one sequence of eight square-wave electric voltage pulses with a magnitude of about 1000 V/cm and a duration of about 100 μs between at least two exemplary electrodes 104a and 104b (and/or two exemplary electrodes 108a and 108b).

In an exemplary embodiment, step 204 may further include generating an exemplary pulsed electric field all over an exemplary target region; allowing for electroporation of all exemplary target cells therein. In an exemplary embodiment, step 204 may further include generating an exemplary pulsed electric field between every two electrodes of at least one of first set of electrodes 104, second set of electrodes 108, and combinations thereof by generating an exemplary pulsed electric field between every possible pair of electrodes of at least one of first set of electrodes 104, second set of electrodes 108, and combinations thereof. In an exemplary embodiment, generating an exemplary pulsed electric field between every two electrodes of at least one of first set of electrodes 104, second set of electrodes 108, and combinations thereof may include generating an exemplary pulsed electric field in a plurality of directions in an exemplary target region; thereby, resulting in electrical stimulation of all parts of an exemplary target region.

In an exemplary embodiment, generating an exemplary pulsed electric field between every two electrodes of at least one of first set of electrodes 104, second set of electrodes 108, and combinations thereof may include periodically changing a connection of at least one of first set of electrodes 104, second set of electrodes 108, and combinations thereof to two poles 132a and 132b of electrical pulse generator 132. In an exemplary embodiment, periodically changing an exemplary connection of at least one of first set of electrodes 104, second set of electrodes 108, and combinations thereof to two poles 132a and 132b of electrical pulse generator 132 may include periodically substituting connection of at least one electrode of at least two electrodes 104a and 104b by a different electrode 104c of first set of electrodes 104. In an exemplary embodiment, substituting connection of at least one electrode of at least two electrodes 104a and 104b by different electrode 104c of first set of electrodes 104 may include electrically disconnecting an exemplary at least one electrode (e.g., 104a) of at least two electrodes 104a and 104b from first pole 132a of electrical pulse generator 132 and electrically connecting different electrode 104c of plurality of electrodes 104 to first pole 132a of electrical pulse generator 132.

In an exemplary embodiment, periodically changing an exemplary connection of at least one of first set of electrodes 104, second set of electrodes 108, and combinations thereof to two poles 132a and 132b of electrical pulse generator 132 may include stepwise changing an exemplary connection of at least one of first set of electrodes 104, second set of electrodes 108, and combinations thereof to two poles 132a and 132b of electrical pulse generator 132 during a plurality of time steps. In an exemplary embodiment, each time step of an exemplary plurality of time steps may include a time interval between about 0.5 second and about 5 seconds.

In an exemplary embodiment, method 200 may be performed for a pre-determined period of time until a therapeutical target, for example, complete destruction of an exemplary plurality of cells including a plurality of cancer cells may be achieved. In an exemplary embodiment, an exemplary pre-determined period of time may depend on an exemplary therapeutical target. In an exemplary embodiment, an exemplary pre-determined period of time may depend on at least one of a location of an exemplary target region, a number of electrodes of first set of electrodes 104 and second set of electrodes 108, an intensity of an exemplary generated pulsed electric field among first set of electrodes 104, and/or second set of electrodes 108, and combinations thereof. In an exemplary embodiment, an exemplary pre-determined period of time may include a continuous time interval or a plurality of intermittent time intervals. In an exemplary embodiment, an exemplary pre-determined period of time may include an exemplary plurality of time steps. For instance, for ECT, treatment duration cannot be more than about 20 minutes in each treatment session due to a decrease in concentration of an applied chemotherapeutic in whole bloodstream.

In an exemplary embodiment, method 200 may further include a step of injecting a drug or a therapeutical substance from a reservoir embedded in tubular probe 100 or using an injection syringe into a location in the vicinity of an exemplary zone containing an exemplary plurality of cells. In an exemplary embodiment, an exemplary injected drug or therapeutical substance may penetrate into electroporated exemplary plurality of target cells; thereby, resulting in treating an exemplary plurality of target cells.

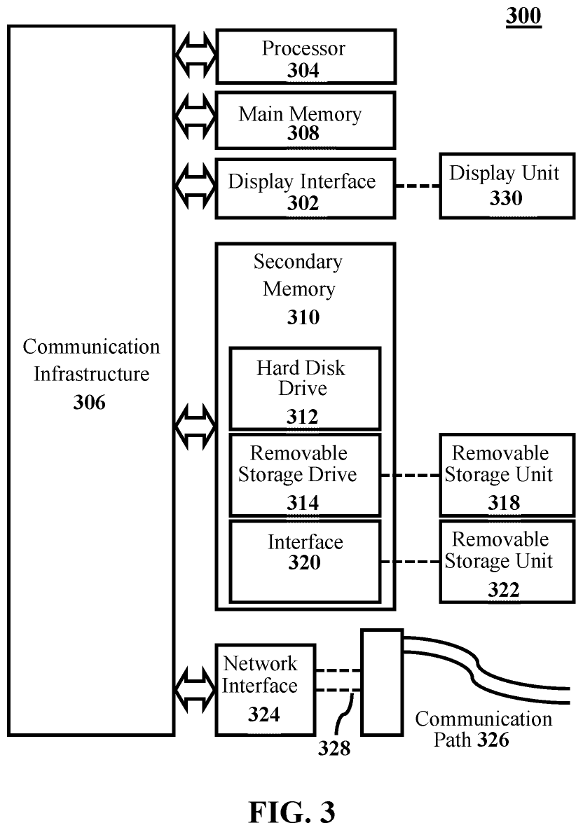

FIG. 3 shows an example computer system 300 in which an embodiment of the present disclosure, or portions thereof, may be implemented as computer-readable code, consistent with one or more exemplary embodiments of the present disclosure. For example, computer system 300 may include an example of processing unit 134, and step 204 of flowchart presented in FIG. 2 may be implemented in computer system 300 using hardware, software, firmware, tangible computer readable media having instructions stored thereon, or a combination thereof and may be implemented in one or more computer systems or other processing systems. Hardware, software, or any combination of such may embody any of the modules and components in FIGS. 1E and 2.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores”.

An embodiment of the present disclosure is described in terms of this example computer system 300. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 304 may be a special purpose or a general-purpose processor device. As will be appreciated by persons skilled in the relevant art, processor device 304 may also be a single processor in a multi-core/multiprocessor system, such system operating alone, or in a cluster of computing devices operating in a cluster or server farm. Processor device 304 may be connected to a communication infrastructure 306, for example, a bus, message queue, network, or multi-core message-passing scheme.

In an exemplary embodiment, computer system 300 may include a display interface 302, for example a video connector, to transfer data to a display unit 330, for example, a monitor. Computer system 300 may also include a main memory 308, for example, random access memory (RAM), and may also include a secondary memory 310. Secondary memory 310 may include, for example, a hard disk drive 312, and a removable storage drive 314. Removable storage drive 314 may include a floppy disk drive, a magnetic tape drive, an optical disk drive, a flash memory, or the like. Removable storage drive 314 may read from and/or write to a removable storage unit 318 in a well-known manner. Removable storage unit 318 may include a floppy disk, a magnetic tape, an optical disk, etc., which may be read by and written to by removable storage drive 314. As will be appreciated by persons skilled in the relevant art, removable storage unit 318 may include a computer usable storage medium having stored therein computer software and/or data.

In alternative embodiments, secondary memory 310 may include other similar means for allowing computer programs or other instructions to be loaded into computer system 300. Such means may include, for example, a removable storage unit 322 and an interface 320. Examples of such means may include a program cartridge and cartridge interface (such as that found in video game devices), a removable memory chip (such as an EPROM, or PROM) and associated socket, and other removable storage units 322 and interfaces 320 which allow software and data to be transferred from removable storage unit 322 to computer system 300.

Computer system 300 may also include a communications interface 324. Communications interface 324 allows software and data to be transferred between computer system 300 and external devices. Communications interface 324 may include a modem, a network interface (such as an Ethernet card), a communications port, a PCMCIA slot and card, or the like. Software and data transferred via communications interface 324 may be in the form of signals, which may be electronic, electromagnetic, optical, or other signals capable of being received by communications interface 324. These signals may be provided to communications interface 324 via a communications path 326. Communications path 326 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 318, removable storage unit 322, and a hard disk installed in hard disk drive 312. Computer program medium and computer usable medium may also refer to memories, such as main memory 308 and secondary memory 310, which may be memory semiconductors (e.g. DRAMs, etc.).

Computer programs (also called computer control logic) are stored in main memory 508 and/or secondary memory 310. Computer programs may also be received via communications interface 324. Such computer programs, when executed, enable computer system 300 to implement different embodiments of the present disclosure as discussed herein. In particular, the computer programs, when executed, enable processor device 304 to implement the processes of the present disclosure, such as the operations in method 200 illustrated by FIG. 2, discussed above. Accordingly, such computer programs represent controllers of computer system 300. Where an exemplary embodiment of method 200 is implemented using software, the software may be stored in a computer program product and loaded into computer system 300 using removable storage drive 314, interface 320, and hard disk drive 312, or communications interface 324.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

### Industrial Applicability

Disclosed herein is a device, system, and method using thereof for electrically stimulation of an internal target region of a living body without any injuries to adjacent tissues. An exemplary probe disclosed herein has a tubular shape allowing for non-invasive access to internal parts of body, especially, hard to access areas, such as abdominal cavity. Exemplary probe, system, and method disclosed here can be used for in-vivo electroporation of a plurality of target cells in sensitive and/or difficult-to-access regions of a living body, such as cancer cells of an internal tumor or cancer cells remained in margins of a resected tumor. Using an exemplary probe, limited areas inside a living body can be exposed to an electric field (e.g., a pulsed electric field) by applying an electrical signal to electrodes of an exemplary probe when is inserted into an exemplary target region. An exemplary probe is made of a silicone tube with conductive electrodes in at least two forms of rings there around and/or flat wires at front surface of an exemplary silicone tube. An exemplary probe is suitable for use in cases where an infection or cancer cells are in inner walls of rectum or vagina. In addition, an exemplary probe can be efficient for colon cancers in any cases where a patient needs electroporation. Regardless of whether an electroporation treatment is palliative, presurgical, or is done for clearing possible involvement in anastomotic area, and also there is no access to a desired area from inside of abdominal cavity, colon wall can be subjected to electrochemotherapy by inserting exemplary probe into abdominal cavity from lumen area.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A tubular probe for non-invasive electrically stimulation of a plurality of target cells in an internal target region of a living body, the tubular probe comprising:
a support tube comprising a plurality of openings thereon, the support tube comprising a distal end and a proximal end, the distal end of the support tube configured to be inserted into the internal target region via guiding the proximal end;
a plurality of circumferential rings defining a first set of electrodes, the plurality of circumferential rings positioned around the support tube in an axially spaced apart relation from each other along the support tube, the first set of electrodes configured to be put in contact with the internal target region;
a head part attached to the distal end of the support tube, the head part comprising:
an electrode substrate engaged to the distal end of the support tube, the electrode substrate comprising at least two apertures; and
at least two U-shaped wires protruded out from the respective at least two apertures, the at least two U-shaped wires defining a second set of electrodes configured to be put in contact with the internal target region; and
a plurality of electrical connectors passing through the support tube, the plurality of electrical connectors comprising:
a first set of electrical connectors configured to connect the first set of electrodes to an electrical stimulator device, each respective electrical connector of the first set of electrical connectors comprising an electrically conductive line, a distal end of the electrically conductive line passed through a respective opening of the plurality of openings and attached to a respective circumferential ring of the plurality of circumferential rings, the distal end of the electrically conductive line fastening the respective circumferential ring around the support tube; and
a second set of electrical connectors configured to connect the second set of electrodes to the electrical stimulator device, each respective electrical connector of the second set of electrical connectors comprising an electrically conductive line comprising a distal end attached to a respective U-shaped wire of the at least two U-shaped wires,
wherein a respective proximal end of each electrically conductive line is in connection with, or capable of connection with, the electrical stimulator device.

2. The tubular probe of claim 1, wherein the support tube comprises an elongated tube made of a biocompatible flexible material.

3. The tubular probe of claim 1, wherein the support tube comprises a biocompatible polymeric tube.

4. The tubular probe of claim 1, wherein the support tube comprises a tube with a diameter in a range of 0.5 cm to 3 cm.

5. The tubular probe of claim 1, wherein each opening of the plurality of openings comprises a hole on the support tube with a diameter in a range of 0.1 cm to 0.5 cm, each two adjacent openings arranged within a distance in a range of 0.5 cm to 1.5 cm from each other.

6. The tubular probe of claim 1, wherein each electrode of the first set of electrodes comprises an electrically conductive biocompatible ring with an internal diameter in a range of 0.5 cm to 3 cm and an external diameter in a range of 0.7 cm to 3.5 cm.

7. The tubular probe of claim 1, wherein each U-shaped wire of the at least two U-shaped wires comprises a biocompatible electrically conductive wire with a diameter in a range of about 0.5 mm to about 3 mm.

8. The tubular probe of claim 1, wherein the at least two U-shaped wires are arranged in parallel to each other with a distance in a range of 0.5 cm to 1.5 cm between each two adjacent U-shaped wires of the at least two U-shaped wires.

9. A system for in-vivo electroporation of a plurality of target cells in an internal target region of a living body, the system comprising:
a tubular probe configured to transfer a pulsed electric field to the plurality of target cells, the tubular probe comprising:
a support tube comprising a plurality of openings thereon, the support tube comprising a distal end and a proximal end, the distal end of the support tube configured to be inserted into the internal target region via guiding the proximal end;
a plurality of circumferential rings defining a first set of electrodes, the plurality of circumferential rings positioned around the support tube in an axially spaced apart relation from each other along the support tube, the first set of electrodes configured to be put in contact with the internal target region;
a head part attached to the distal end of the support tube, the head part comprising:
an electrode substrate engaged to the distal end of the support tube, the electrode substrate comprising at least two apertures; and
at least two U-shaped wires protruded out from the respective at least two apertures, the at least two U-shaped wires defining a second set of electrodes configured to be put in contact with the internal target region; and
a plurality of electrical connectors passing through the support tube, the plurality of electrical connectors comprising:
a first set of electrical connectors, each respective electrical connector of the first set of electrical connectors comprising an electrically conductive line comprising a distal end and a proximal end, the respective distal end passed through a respective opening of the plurality of openings and attached to a respective circumferential ring of the plurality of circumferential rings, the distal end of the electrically conductive line fastening the respective circumferential ring around the support tube; and
a second set of electrical connectors, each respective electrical connector of the second set of electrical connectors comprising an electrically conductive line comprising a distal end and a proximal end, the respective distal end attached to a respective U-shaped wire of the at least two U-shaped wires,
an electrical pulse generator configured to apply a pulsed electric field between at least two electrodes of at least one of the first set of electrodes, the second set of electrodes, and combinations thereof, each respective proximal end of each electrically conductive line of the at least two electrodes being connected to a different pole of two poles of the electrical pulse generator; and
a processing unit electrically connected to the electrical pulse generator, the processing unit comprising:
a memory having processor-readable instructions stored therein; and
a processor configured to access the memory and execute the processor-readable instructions, which, when executed by the processor configures the processor to perform a method, the method comprising:
inducing electroporation to the plurality of target cells by generating the pulsed electric field between the at least two electrodes of at least one of the first set of electrodes, the second set of electrodes, and combinations thereof inside the internal target region via applying at least one sequence of electric voltage pulses between the at least two electrodes utilizing the electrical pulse generator.

10. The system of claim 9, wherein applying the at least one sequence of electric voltage pulses between the at least two electrodes comprises applying at least one sequence of eight square-wave electric voltage pulses with a magnitude in a range of 500 V/cm to 1500 V/cm and a duration of 100 μs between the at least two electrodes.

11. The system of claim 9, wherein the method further comprises generating the pulsed electric field between every two electrodes of at least one of the first set of electrodes, the second set of electrodes, and combinations thereof by periodically changing a connection of at least one of the first set of electrodes, the second set of electrodes, and combinations thereof to the two poles of the electrical pulse generator.

12. The system of claim 11, wherein periodically changing the connection of at least one of the first set of electrodes, the second set of electrodes, and combinations thereof to the two poles of the electrical pulse generator comprises periodically substituting connection of at least one electrode of the at least two electrodes by a different electrode of at least one of the first set of electrodes and the second set of electrodes, substituting connection of the at least one electrode of the at least two electrodes by the different electrode of at least one of the first set of electrodes and the second set of electrodes comprises:
electrically disconnecting the at least one electrode of the at least two electrodes from a first pole of the electrical pulse generator; and
electrically connecting the different electrode of at least one of the first set of electrodes and the second set of electrodes to the first pole of the electrical pulse generator.

13. The system of claim 11, wherein periodically changing the connection of at least one of the first set of electrodes, the second set of electrodes, and combinations thereof to the two poles of the electrical pulse generator comprises stepwise changing the connection of at least one of the first set of electrodes, the second set of electrodes, and combinations thereof to the two poles of the electrical pulse generator during a plurality of time steps.

14. The system of claim 13, wherein each time step of the plurality of time steps comprises a time interval between 0.5 second and 5 seconds.

15. The system of claim 9, wherein the support tube comprises an elongated biocompatible flexible tube.

16. The system of claim 9, wherein the support tube comprises a tube with a diameter in a range of 0.5 cm to 3 cm.

17. The system of claim 9, wherein each opening of the plurality of openings comprises a hole on the support tube with a diameter in a range of 0.1 cm to 0.5 cm, each two adjacent openings arranged within a distance in a range of 0.5 cm to 1.5 cm from each other.

18. The system of claim 9, wherein each electrode of the first set of electrodes comprises an electrically conductive biocompatible ring with an internal diameter in a range of 0.5 cm to 3 cm and an external diameter in a range of 0.7 cm to 3.5 cm.

19. The system of claim 9, wherein each U-shaped wire of the at least two U-shaped wires comprises a biocompatible electrically conductive wire with a diameter in a range of about 0.5 mm to about 3 mm.

20. The system of claim 1, wherein the at least two U-shaped wires are arranged in parallel to each other with a distance in a range of 0.5 cm to 1.5 cm between each two adjacent U-shaped wires of the at least two U-shaped wires.
